(19) United States
(12) Patent Application Publication
(10) Pub. No.: US 2012/0089010 A1
(43) Pub. Date: Apr. 12, 2012

(54) HYPERPOLARIZED CONTRAST AGENT DISPENSER FOR MAGNETIC RESONANCE IMAING

(75) Inventors: Christoph Leussler, Hamburg (DE); Peter Boernert, Hamburg (DE); Daniel Wirtz, Hamburg (DE); Bernd David, Huettblek (DE); Holger Eggers, Ellerhoop (DE); Jochen Keupp, Rosengarten (DE); Johannes Adrianus Overweg, Uelzen (DE); Rainer Eckart, Hamburg (DE)

(73) Assignee: KONINKLIJKE PHILIPS ELECTRONICS N.V., EINDHOVEN (NL)

(21) Appl. No.: 13/377,831

(22) PCT Filed: Jun. 23, 2010
(86) PCT No.: PCT/IB10/52864

§ 371 Date: Dec. 13, 2011

(30) Foreign Application Priority Data
Jun. 26, 2009 (EP) 09163862.7

(51) Int. Cl. A61B 5/055

(52) U.S. Cl. 600/413; 600/420

(57) ABSTRACT

A dispenser (132), a magnetic resonance imaging system (100), and a method for using hyperpolarized contrast agent (304) during a magnetic resonance imaging examination. The dispenser comprises an attachment component (136) for a face piece (138). The face piece is adapted for receiving the surface of a subject (114) such that when the subject inhales hyperpolarized contrast agent enters the respiratory system of the subject. The dispenser further comprises a reservoir (300) adapted for receiving the hyperpolarized contrast agent. The dispenser further comprises a gas flow (406) tube connected to the attachment component and a vaporizer (406, 408, 412, 510, 602, 606) for vaporizing the hyperpolarized contrast agent in the gas flow tube into a hyperpolarized vapor. The dispenser further comprises a controller (402) for controlling when the vaporizer vaporizes the hyperpolarized contrast agent.

### FIELD OF THE INVENTION

The invention relates to a dispenser, a magnetic resonance imaging system, and a method for using hyperpolarized contrast agent in magnetic resonance imaging, in particular the invention relates to equipment and method of using of vaporized hyperpolarized contrast agent during a magnetic resonance imaging examination.

### BACKGROUND OF THE INVENTION

A static magnetic field is used by MRI scanners to align the nuclear spins of atoms as part of the procedure for producing images within the body of a patient or subject. This large static magnetic field is referred to as the B₀ field. It is commonly known that increasing the strength of the Bₒ field used for performing an MRI scan offers the opportunity of increasing the spatial resolution and contrast resolution of the diagnostic images. This increase in resolution and contrast is beneficial for physicians using the MRI image to diagnose a patient.

Magnetic resonance imaging systems typically are used to image the concentrations of protons, or hydrogen atoms, in a subject. As a result magnetic resonance imaging systems have been very useful for imaging the soft tissues of a subject. The structure of the lungs and its vascularization is also of interest. However the lungs are mostly air, which cannot be easily imaged by magnetic resonance imaging. The use of injected magnetic resonance imaging contrast agents is known.

However, it typically takes about 15 seconds for an intravenously injected substance to reach the lung vessels. Many hyperpolarized contrasts agents very short relaxation time such that a substantial portion of the contrast agent will lose its polarization within these 15 seconds. For example, hyperpolarized ¹³C has a T₁ relaxation time of the order of 60 seconds. The journal article Ishii et. al., Magnetic Resonance in Medicine 57: 459-463 (2007) demonstrates the use of a hyperpolarized ¹³C angiography for evaluating pulmonary perfusion. Hyperpolarized hydroxyethylpropionate was injected into the femoral artery of pigs in this experimental study.

### SUMMARY OF THE INVENTION

The invention provides a dispenser for dispensing a hyperpolarized vapor, a magnetic resonance imaging system, and a method of acquiring a magnetic resonance image in the independent claims. Embodiments of the invention are given in the dependent claims.

Embodiments of the invention address this and other problems by vaporizing a liquid or solid hyperpolarized contrast agent. A dispenser is used to vaporize the hyperpolarized contrast agent into a mist or vapors which are then inhaled by the subject. This delivers the hyperpolarized contrast agent directly to the lungs and increases the effectiveness of the hyperpolarized contrast agent.

The invention can be applied to any MRI system that is adapted for hyperpolarized imaging. More specifically, it is applicable in lung imaging for the detection of lesions and abnormal functional processes. A couple of substances could potentially be used for polarization. Among different metabolites which undergo further digestion, enzymes, therapeutic agents or targeted agents could be hyperpolarized to study their biodistribution and fate in-vivo by means of fast spectroscopic ¹³C imaging

As used herein, the term coil refers to a radio frequency antenna that is used to acquire magnetic resonance imaging data. The term coil may refer to the case where a single antenna performs both the transmission and reception of radio frequency signals during the acquisition of magnetic resonance imaging data or it may refer to a transmit and an receive antenna. Moreover an array configuration for either case mentioned consisting of a multitude of single elements is considered.

As used herein, the term hyperpolarized contrast agent refers to a hyperpolarized material that is used as a contrast agent during the acquisition of magnetic resonance imaging data. The hyperpolarized contrast agent may be in liquid or solid form before it is vaporized. Before use, the hyperpolarized contrast agent may be polarized using a process such as Dynamic Nuclear Polarization (DNP) or any other suitable process. The polarized nuclei of the hyperpolarized contrast agent, with the exception of noble gases such as He and Xe, may be embedded into a biocompatible molecule. Isotopes that may be used to make hyperpolarized contrast agents include: ⁷Li, ¹³C, ¹⁴N, ¹⁵N, ¹⁷O, ¹⁹F, ²³Na, ³¹P. Since it is beneficial to embedded the isotope into a biocompatible molecule ¹³C is of particular interest, due to the presence of carbon in many of the body's molecules. ¹⁴N and ¹⁷O also offer the possibility of being more easily integrated into biocompatible molecules.

Magnetic Resonance Imaging (MRI) data is defined herein as being the recorded measurements of radio frequency signals emitted by atomic spins by the antenna of a Magnetic resonance apparatus during a magnetic resonance imaging scan. A Magnetic Resonance Imaging (MRI) image is defined herein as being the reconstructed two or three dimensional visualization of anatomic data contained within the magnetic resonance imaging data. This visualization can be performed using a computer system.

A computer system is defined herein as a computer or a collection of computers. For magnetic resonance imaging, a single computer may be used to operate and perform analysis of the magnetic resonance imaging data. However, this functionality is often distributed across many different computers, and the magnetic resonance imaging data may be stored for analysis by a computer or computer system later.

The invention provides for a dispenser for dispensing a hyperpolarized vapor to a subject during a magnetic resonance imaging examination. The dispenser comprises an attachment component for a face piece adapted to receive a surface of a subject such that when the subject inhales the hyperpolarized vapor enters the respiratory system of the subject. The dispenser further comprises a reservoir adapted for receiving a hyperpolarized contrast agent. The dispenser further comprises a gas flow tube connected to the attachment component. The dispenser further comprises a vaporizer for vaporizing the hyperpolarized contrast agent in the gas flow into the hyperpolarized vapor. The dispenser further comprises a controller for controlling when the vaporizer vaporizes the hyperpolarized contrast agent. The attachment component allows a face piece or a face piece in a tube to be attached to the dispenser. This is beneficial, because more than one patient may be treated using a magnetic resonance imaging apparatus system. In this case it would be beneficial to use disposable face pieces which can be removed and cleaned.

The reservoir is adapted for receiving a hyperpolarized contrast agent, the latter is either in a solid or a liquid form. There is a gas flow tube where the hyperpolarized contrast is vaporized. The controller controls when the vaporizer vaporizes the hyperpolarized contrast agent. This is particularly beneficial, because the hyperpolarized contrast agents can be extremely expensive and only very small amounts may be available at a time. This minimizes the amount of hyperpolarized contrast agent that is used during the examination. The controller could operate in several different ways, for instance it could receive a signal from the magnetic resonance imaging system when to allow the dispensing of the hyperpolarized contrast agent. The controller could also operate by having some sort of sensor system where the breathing cycle of the subject is detected. For instance the pressure or the flow or the temperature could be measured at some point in the face piece or gas flow tube or tube connecting the attachment component to a face piece in order to detect when the subject is breathing. The controller could also by receiving a signal from the magnetic resonance imaging system and by using a sensor system.

In an embodiment of the invention, the dispenser further comprises a nuclear magnetic resonance coil adjacent to the reservoir. The nuclear magnetic resonance coil is adapted for measuring the degree of polarization of the hyperpolarized contrast agent when connected to a nuclear magnetic resonance apparatus. This is advantageous because the degree of polarization of the hyperpolarized contrast agent can be measured. A nuclear magnetic resonance apparatus is defined herein as a apparatus which uses nuclear magnetic resonance to measure the material properties of a sample.

In an embodiment of the invention, the reservoir is adapted for storing the hyperpolarized contrast agent in liquid form. This is advantageous, because the hyperpolarized contrast agent can be generated in a solid state form and then is liquefied and placed into the reservoir and then it is vaporized.

In an embodiment the reservoir is adapted for storing the hyperpolarized contrast agent in solid form. The dispenser further comprises a heater adapted for liquefying the hyperpolarized contrast agent before vaporization. This embodiment is advantageous, because the lifetime of the hyperpolarized contrast agent is longer when the hyperpolarized contrast agent remains in solid form. During the hyperpolarization process the hyperpolarized contrast agent may be enriched with chemicals that facilitate the polarization process. These chemicals may be toxic. When liquid hyperpolarized contrast agents are prepared, the solid hyperpolarized contrast agent is melted and any toxic chemicals are filtered out before administering them to a subject. If a solid contrast agent is used, it may be beneficial to incorporate a filter into either into the dispenser or into a cartridge that fits into the reservoir of the dispenser that is used for the hyperpolarized contrast agent. The solid hyperpolarized contrast agent would be melted, pass through a filter to remove any toxic chemicals and then be vaporized.

In another embodiment the heater is adapted for melting the hyperpolarized contrast agent at a rate equal to the rate at which it is vaporized. This embodiment is particularly advantageous, because the hyperpolarized contrast agent leaves the solid form at exactly the right rate. This means that the hyperpolarized contrast agent will have a longer lifetime.

In another embodiment the dispenser further comprises a breathing sensor adapted for detecting inhalation and exhalation by the subject. The controller is adapted for controlling the vaporizer such that the hyperpolarized contrast agent is vaporized only during inhalation by the subject. This embodiment is advantageous, because the hyperpolarized contrast agent is not wasted. The breathing sensor could be implemented in a variety of ways for instance the gas flow through gas flow tube could be measured, a pressure sensor could be used to detect when the subject is not breathing or even a temperature sensor could be used by determining the temperature of the gas exiting the respiratory system of the subject.

The breathing sensor can be implemented using a temperature sensor, a gas flow sensor, and/or pressure sensor.

In another embodiment the controller is adapted for receiving instructions from the computer of a magnetic resonance imaging system. The computer system is adapted for instructing the controller when to allow the vaporizer to vaporize the hyperpolarized contrast agent. This embodiment is advantageous, because the hyperpolarized contrast agent is only administered when it is necessary during the imaging sequence. If the lifetime of the hyperpolarized contrast agent is extremely short unless the magnetic resonance imaging system is acquiring magnetic resonance imaging data for the purpose of imaging the concentration of the hyperpolarized contrast agent, it is not useful to dispense the hyperpolarized vapor.

In another embodiment the hyperpolarized contrast agent is vaporized using a carburetor. A carburetor is understood herein as a device which sprays a liquid into a gas flow for the purpose of evaporation or vaporization. This embodiment is advantageous, because the liquid hyperpolarized contrast agent can be sprayed into the gas flow and vaporized.

In another embodiment, the hyperpolarized contrast agent is vaporized using a piezoelectric vibrator. This could be in the form of a reservoir which is filled with the hyperpolarized contrast agent, or it could be in the form of a piezoelectric nozzle which sprays the hyperpolarized contrast agent into the gas flow tube.

In another embodiment the reservoir is adapted for storing the hyperpolarized contrast agent in solid form. The vaporizer comprises a chopper adapted for chopping the hyperpolarized contrast agent into particles small enough that the particles melt or are vaporized before being inhaled by the subject. This embodiment is advantageous, because it maximizes the lifetime of the hyperpolarized contrast agent before inhalation by the subject.

In another embodiment the reservoir is adapted for receiving a cartridge containing the hyperpolarized contrast agent. This is an advantageous embodiment, because the cartridge provides a convenient way of bringing the hyperpolarized contrast agent into the dispenser.

In another embodiment the dispenser further comprises a cartridge holder adapted for holding at least two cartridges. The cartridge holder is adapted for automatically changing which cartridge is mounted in the reservoir. This embodiment is advantageous, because the hyperpolarized contrast agent can be prepared for use over an extended period of time. During the course of an examination or during the course of several different examinations, the hyperpolarized contrast agent can be loaded into the reservoir automatically.

In another embodiment the dispenser further comprises a bypass valve adapted for selecting if gas flow through the gas flow tube is directed to the face piece or is directed to a cleaning outlet which is used for venting during a self-cleaning procedure. The control system is adapted for controlling the bypass valve in order to direct gas flow to the cleaning outlet during a self-cleaning procedure. This embodiment is advantageous, because the system can be used over an extended period of time if the system does not need to be taken apart and cleaned.

In another embodiment at least one portion of the gas flow tube has an electric heater adapted for re-vaporizing condensate of the hyperpolarized vapor. This embodiment is advantageous, because the hyperpolarized material could condense on the gas flow tube. This embodiment makes more efficient use of the hyperpolarized contrast agent and can be used in conjunction with a self-cleaning procedure.

In another embodiment, the dispenser, face piece and/or cartridges may have an RFID tag for identification.

In another embodiment, the dispenser, face piece, and/or cartridge may have a bar code.

In another embodiment, the dispenser, cartridge, and/or face piece are disposable.

In another aspect, the invention provides for a magnetic resonance imaging system that is adapted for acquiring magnetic resonance imaging images comprising a magnet for generating a magnetic field for orientating the magnetic spins of nuclei. The magnet may be a superconducting, a resistive magnet or a combination of superconducting and resistive magnet. A toroidal magnet or a so-called open magnet may also be used. The magnetic resonance imaging system further comprises a radio frequency system comprising a first coil calibrated for acquiring magnetic resonance imaging data of protons. The radio frequency system further comprises a second coil calibrated for acquiring magnetic resonance imaging data for the hyperpolarized contrast agent. It is understood that the first coil and the second coil may be integrated into a single coil assembly or may share common parts. Essentially the first coil and the second coil can be the same coil. The magnetic resonance imaging system further comprises a magnet field gradient coil for spatially encoding and manipulating the orientation of the magnetic spins of nuclei. The magnetic resonance imaging system further comprises a magnet field gradient power supply for supplying current to the magnetic field gradient coil. The magnetic resonance imaging system further comprises a computer system for constructing images from the magnetic resonance imaging data and for controlling the operation of the magnetic resonance imaging system. It is understood herein that a computer system can refer to one or more computers embedded systems or controllers. As was mentioned before, in some cases one computer controls the magnetic resonance imaging system and another computer or a computer system reconstructs the images from the data. The magnetic resonance imaging system further comprises a dispenser according to an embodiment of the invention.

In another embodiment the reservoir of the dispenser is mounted with a location fixed relative to the magnetic field of the magnet. The location is chosen such that the magnetic field of the magnet preserves the hyperpolarization of the hyperpolarized contrast agent. This embodiment is advantageous, because the magnetic field helps to increase the lifetime of the hyperpolarized contrast agent.

In another aspect the invention provides for a method of acquiring a magnetic resonance imaging system of the subject using a magnetic resonance imaging system according to an embodiment of the invention. The method comprises the step of acquiring a first set of magnetic resonance imaging data from within a region of interest of the subject using the first coil. The first coil is calibrated for acquiring proton magnetic resonance imaging data. Proton magnetic resonance imaging data refers to the acquisition of magnetic resonance imaging data using hydrogen atoms. Within a subject, the hydrogen molecules will be incorporated into molecules such as water, fat, or body tissues.

The method further comprises calibrating a second radio frequency coil with the computer system using the first set of magnetic resonance imaging data and a model. This step is particularly advantageous, because the lifetime of the hyperpolarized contrast agent is extremely small, it would not necessarily be practical to calibrate the second coil but instead it would be more practical to use a model in conjunction with the first set of magnetic resonance imaging data to construct the calibration. The model could be constructed in several different ways; it could be conducted empirically by correlating magnetic resonance imaging data from the first coil with measurements taken making the second radio frequency coil. The calibration could also be constructed using a radio frequency model of the subject and modeling what the likely response in a frequency range would be using information obtained about the subject using the first coil. The method further comprises administering the hyperpolarized vapor to the subject using the dispenser. The method further comprises acquiring a second set of magnetic resonance imaging data in the region of interest using the second coil. The method further comprises constructing a magnetic resonance imaging image from the second set of magnetic resonance imaging data using the computer system. It is understood herein that the computer system is not necessarily in the same physical location as the magnetic resonance imaging system. For instance a collection of computers could be attached to the magnetic resonance imaging system by a network connection.

### BRIEF DESCRIPTION OF THE DRAWINGS

In the following preferred embodiments of the invention will be described, by way of example only, and with reference to the drawings in which:

FIG. 1 illustrates a magnetic resonance imaging system according to an embodiment of the invention,

FIG. 2 shows a flow chart of a method according to an embodiment of the invention,

FIG. 3 illustrates a dispenser according to an embodiment of the invention,

FIG. 4 illustrates a dispenser according to a further embodiment of the invention,

FIG. 5 illustrates a dispenser according to a further embodiment of the invention,

FIG. 6 illustrates a dispenser according to a further embodiment of the invention,

FIG. 7 illustrates a dispenser with a cartridge holder according to an embodiment of the invention,

FIG. 8 illustrates a dispenser with a cartridge holder according to a further embodiment of the invention,

FIG. 9 illustrates a dispenser with a cartridge holder according to a further embodiment of the invention,

FIG. 10 illustrates a functional block diagram of a magnetic resoance imaging system according to an embodiment of the invention,

FIG. 11 shows a flowchart of a further method according to an embodiment of the invention,

FIG. 12 illustrates a dispenser according to a further embodiment of the invention,

FIG. 13 illustrates a diagram explaining the operation of a bypass valve used during a self cleaning operation,

FIG. 14 illustrates a heating system that is integrated into the gas flow tube according to an embodiment of the invention, and

FIG. 15 shows a flow chart of a further method according to an embodiment of the invention.

### DETAILED DESCRIPTION OF EMBODIMENTS

Like numbered elements in these figures are either identical elements or perform the same function. Elements which have been discussed previously will not necessarily be discussed in later figures if the function is identical.

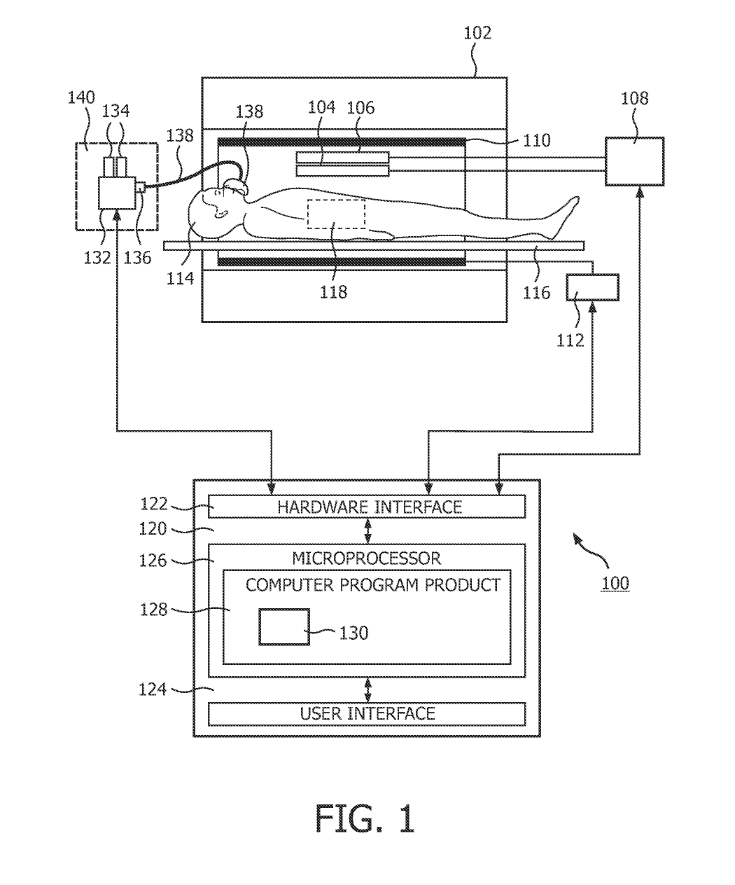

FIG. 1 shows a magnetic resonance imaging system 100 according to an embodiment of the invention. There is a magnet 102 which generates a high magnetic field for aligning the spins of nuclei within the magnet. Inside the bore of the magnet there is a magnetic field gradient coil 110 and a first coil 104 and a second coil 106. The magnetic field gradient coil 110 is connected to a magnetic field gradient coil power supply 112. The first coil 104 and the second coil 106 are connected to a radio frequency transceiver 108. It is understood that separate excitation and receive coils or antennas can be used or combined antennas can also be used. In other embodiments 108 could be a transmitter and a separate receiver. There is also a subject 114 which is sitting upon a subject support 116 within the bore of the magnet 102 also. There is a region of interest 118 for which magnetic resonance imaging data is acquired. Adjacent to the magnet 102 is a dispenser 132 which is adapted for dispensing a hyperpolarized vapor to the subject 114. The dispenser 132 is shown with two cartridges 134. The dispenser 132 also has an attachment component 136 which is used for connecting the face piece 138 to the dispenser 132. The face piece 138 is shown as being over the nose and mouth of the subject 114. In this way the subject 114 can inhale the hyperpolarized vapor during the acquisition of magnetic resonance imaging data. The dispenser 132 is located within a region 140 which has its location fixed relative to the magnetic field of the magnet 102. Region 140 is chosen such that the magnetic field is sufficient to maintain the hyperpolarized material in the cartridges 134.

There is also a computer system 120 shown. The computer system has a hardware interface 122 which is adapted for controlling the magnetic resonance imaging system. The hardware interface is connected to the dispenser 132, the magnetic field gradient coil power supply 112, and the radio frequency transceiver 108. The hardware interface is also connected to a microprocessor 126. The microprocessor has a computer program product 128 which contains machine executable instructions for controlling and operating the magnetic resonance imaging system. A component of the computer program product is model module 130. The model module is a module which is able to use magnetic resonance imaging data acquired using the first coil in order to calculate a calibration for the second coil 106. The computer system 120 is also shown with a user interface 124 where information can be displayed to a user or operator and where also instructions or commands can be received from an operator. As the dispenser would be located in a region of a magnetic field great enough to perform magnetic resonance imaging, the materials and components chosen would be non magnetic in order prevent them from being attracted by the B₀ field.

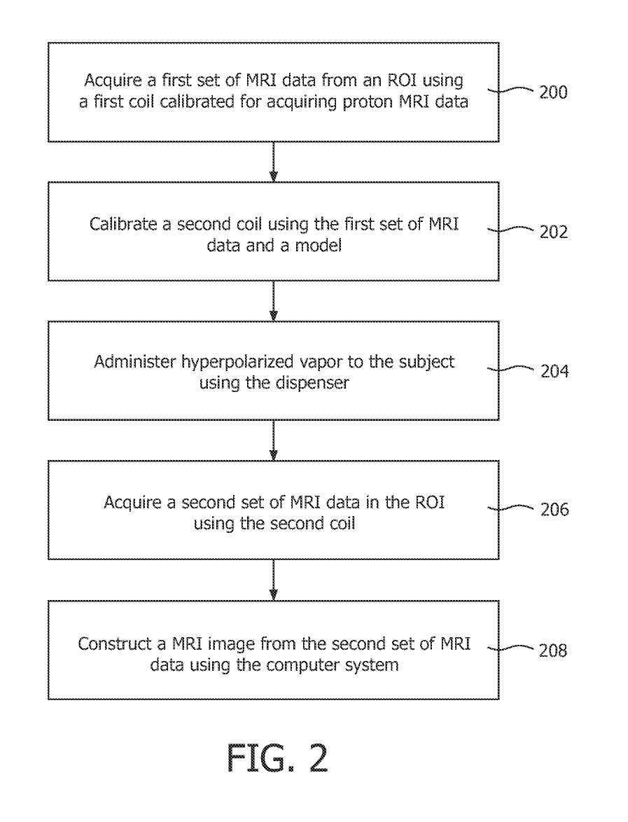

FIG. 2 shows an embodiment of a method according to the invention. In step 200 a first set of MRI data is acquired using the first coil for a region of interest. The first coil is calibrated for acquiring proton magnetic resonance imaging data. In step 202 a second coil is calibrated using the first set of magnetic resonance imaging data and a model. Due to the fast rate of decay of the hyperpolarized material this is advantageous, because normal proton magnetic resonance imaging data is used to determine what the calibration is and this produces the amount of hyperpolarized contrast agent that is necessary and makes the procedure go more rapidly. In step 204 hyperpolarized vapor is administered to the subject using a dispenser. In step 206 a second set of magnetic resonance imaging data region of interest is acquired using the second coil. Finally, in step 208 a magnetic resonance imaging image is constructed from a second set of magnetic resonance imaging data using the computer system. During the construction of the magnetic resonance imaging data, the first set of magnetic resonance imaging data may also be used. In this case the concentration of the hyperpolarized contrast agent is superimposed upon the proton magnetic resonance imaging image.

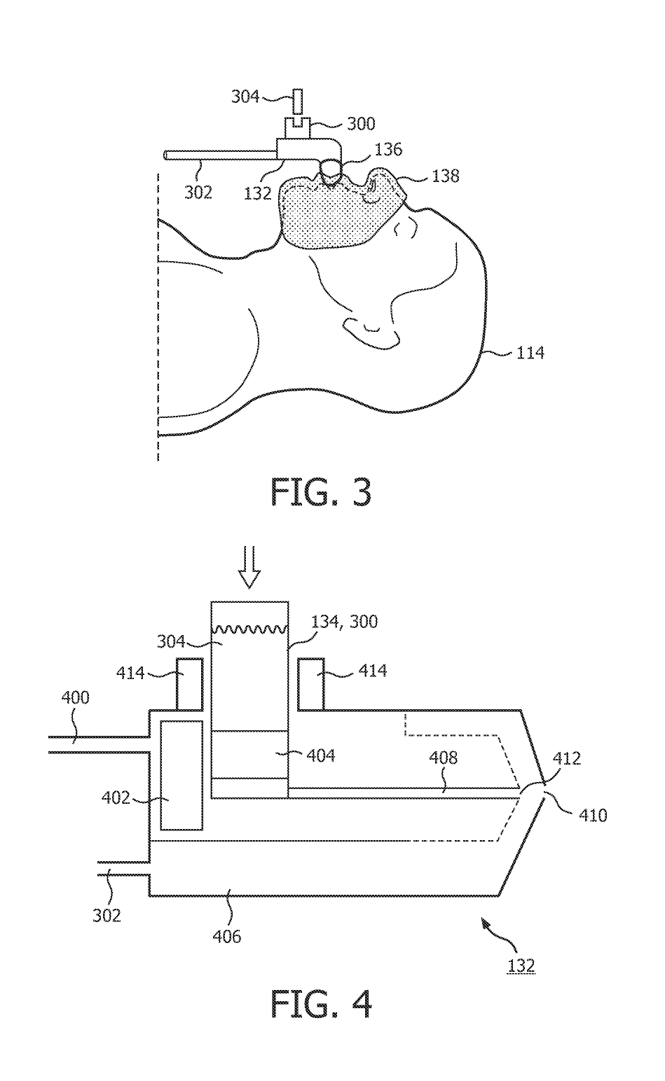

FIG. 3 shows an embodiment of a dispenser according to an embodiment of the invention. Shown is a subject 114 with a face piece 138 over the subject's 114 face. The face piece 138 is attached to an attachment component 136. The attachment component 136 is shown as being a part of a dispenser 132. Entering into the dispenser 132 is a gas inlet 302. This supplies gas to the dispenser 132. Along next to the gas inlet 302 can also be an electrical or optical or wireless connection for controlling the dispenser 132. On top of the dispenser 132 is a reservoir 300. The reservoir is adapted for receiving hyperpolarized contrast agent 304. This is an idealized drawing so the hyperpolarized contrast agent could be in the form of a liquid, a solid or it can also be in the form of a cartridge containing either a liquid or a solid. A wireless connection or an optical connection, such as a fiber optic connection, may be used for remotely controlling the dispenser 132. Since the dispenser may be located within the magnetic field of a magnetic resonance imaging system, it is difficult to employ an electrical connection for remotely controlling the dispenser 132 that is safe at the same time.

FIG. 4 shows a dispenser 132 that uses a carburetion system. There is a cartridge 134 shown containing a liquid hyperpolarized contrast agent 304. The cartridge 134 forms the reservoir 300 in this case. There is a valve 404 which controls the flow of the liquid hyperpolarized contrast agent 304 into a liquid flow tube 408. Adjacent to the cartridge 134 is a support 414, that can also act as or contain a keying mechanism ensuring usage of the right cartridges/agents. In an embodiment where the hyperpolarized contrast agent 304 is instead a solid a heater could be located within the support 414. Adjacent to the valve 404 is shown electronics for the controller and any possible sensors 402. There is an electrical or optical or wireless control line 400 which connects the electronics 402 to a controller or for sending and receiving control signals from the electronics 402. Beneath the liquid flow tube 408 is the gas fluid flow tube 406, where the liquid flow tube 408 enters the gas tube 406 there is a nozzle 412 which sprays the hyperpolarized contrast agent 304 into the gas flow tube 406. A hyperpolarized vapor or mist is formed which then exits the dispenser 132 through an outlet 410 to the attachment component. A transmit and receive nuclear magnetic resonance (NMR) coil may be integrated into the support 414. This NRM coil may be connected to a NMR spectrometer to probe the level of polarization of hyperpolarized material in the reservoir 300. The signal of this NMR probe may also be incorporated into a trigger algorithm controlling the imaging sequence and the operation of the vaporizer. For instance as the level of polarization of the hyperpolarized material decays the dosage of the hyperpolarized material may be increased to compensate.

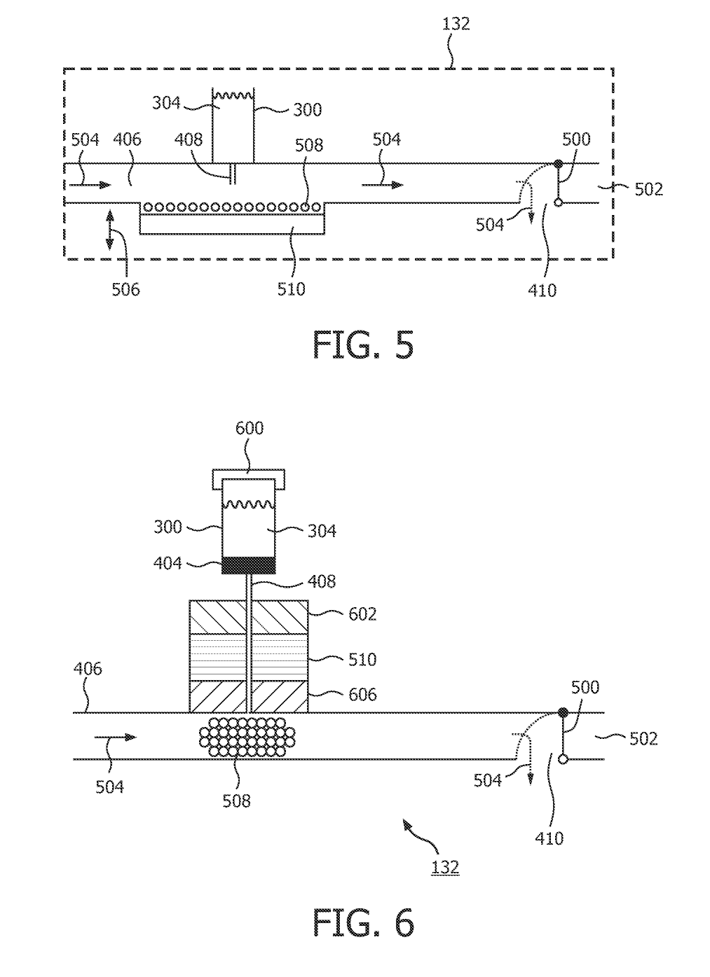

FIG. 5 shows a dispenser 132 that uses a piezoelectric activator 510 to vaporize the hyperpolarized contrast agent 304. In the center of the dispenser 132 is the gas flow tube 406. In the lower section there is a piezoelectric actuator 510. Above the piezoelectric actuator 510 there is a reservoir 300 of hyperpolarized contrast agent 304. The hyperpolarized contrast agent drips onto the piezoelectric actuator 510 through a liquid flow tube 408. The arrows 504 indicate the direction of gas flow through the gas flow tube 406. The arrow labeled 506 indicates the amplitude of vibration of the piezoelectric actuator 510. The piezoelectric actuator 510 vibrates up and down and causes droplets 508 of hyperpolarized contrast agent to form in the gas flow tube. This causes the hyperpolarized contrast agent 304 to be turned into a hyperpolarized vapor. This hyperpolarized contrast agent then exits through the outlet 410 to the attached component. Also shown in this embodiment is a bypass valve 500 which goes to a cleaning outlet 502 that is used during a self-cleaning procedure.

FIG. 6 shows another embodiment of a dispenser 132 that uses a vibrating nozzle 606 that is actuated by a piezoelectric actuator 510. Again there is a gas flow tube 406. The arrows 504 show the direction of airflow through the gas flow tube 406. There is a reservoir 300 filled with hyperpolarized contrast agent 304. In this embodiment a cover 600 is shown on top of the reservoir 300. There is a valve 404 which controls the flow of the hyperpolarized contrast agent 304 through a liquid flow tube 408. The liquid flow tube 408 goes through a support for the piezoelectric actuator 602 and then through the piezoelectric actuator 510 and finally into a vibrating tip 606. The piezoelectric actuator is mounted onto the support for the piezoelectric actuator 602 and the vibrating nozzle 606 is mounted onto the piezoelectric actuator 510. When the piezoelectric actuator 510 is actuated the vibrating tip will move back and forth and essentially vibrate. As the hyperpolarized contrast agent 304 exits the liquid flow tube 408 into the gas flow tube 406, the vibrating nozzle causes droplets 508 of hyperpolarized contrast agent to form in the gas flow tube 406. The vaporized hyperpolarized contrast agent then exits the dispenser through the outlet 410 to the attachment component.

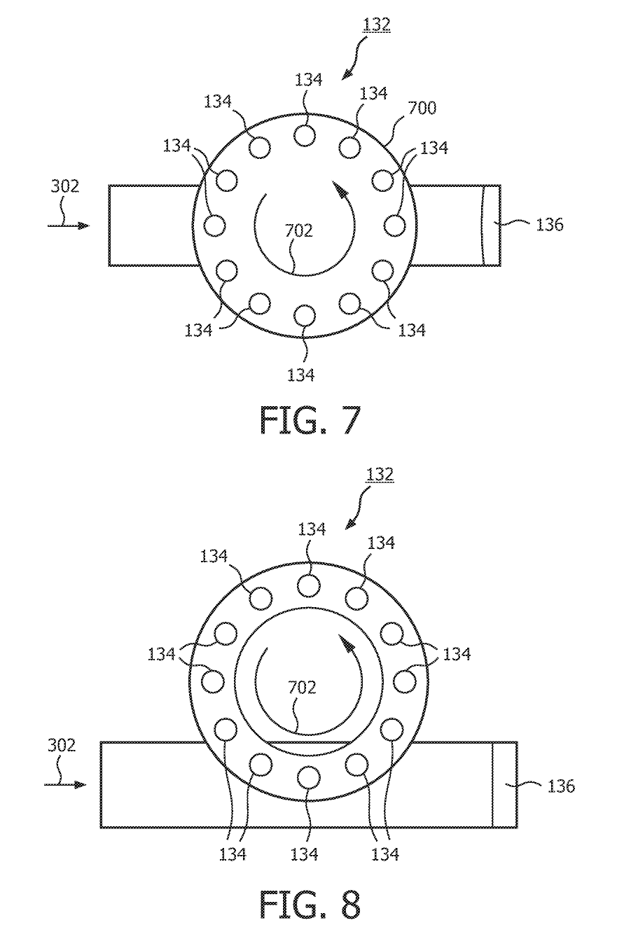

In FIG. 7 a dispenser 132 with a rotating cartridge holder 700 is shown. On one end of the dispenser 132 is a gas inlet 302. On the other end is the attachment component 136. Mounted on top of the dispenser 132 is a cartridge holder 700. In the cartridge holder 700 are multiple cartridges 134. The cartridge holder changes which cartridge is inserted into the dispenser 132 by rotating. The direction of rotation is shown using arrow 702 (which could also be the opposite direction . . . ).

FIG. 8 shows an embodiment of a dispenser 132 with a cartridge holder 700 similar to that shown in FIG. 7. In FIG. 7 the axis of rotation of the cartridge holder 700 was directly over the dispenser 132. In FIG. 8 the axis of rotation does not pass through the dispenser 132. Apart from this, the function of the embodiment shown in FIG. 8 and FIG. 7 is identical.

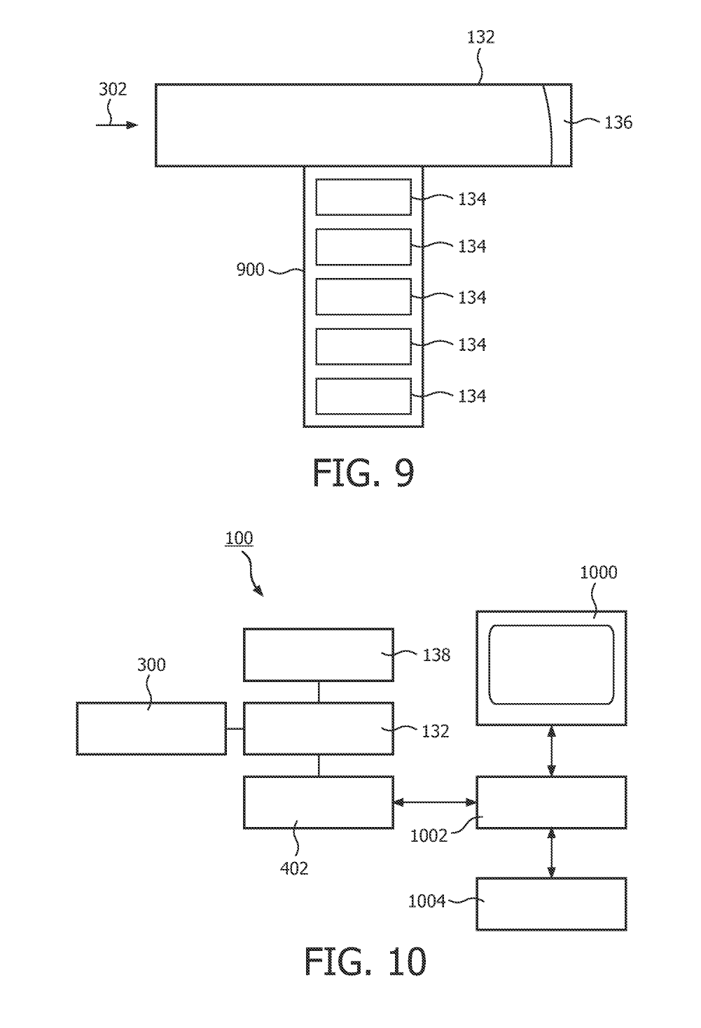

FIG. 9 shows an embodiment of a dispenser with an inner feed cartridge holder 900. On one end of the dispenser is an inlet 302 and on the other side is the attachment component 136. The cartridge holder 900 contains places for individual cartridges 134 of hyperpolarized contrast agent. The cartridge holder moves in a linear fashion and brings each cartridge 134 into position to enable to be used by the dispenser 132.

FIG. 10 shows a functional block diagram of a magnetic resonance imaging system 100 according to an embodiment of the invention. There is a magnetic resonance imaging spectrometer 1002 that is connected to a magnetic resonance imaging magnet and magnetic field gradients 1004. There is a console 1000 or computer system which is used to control and analyze the data coming from the magnetic resonance imaging system 100. The magnetic resonance imaging spectrometer 1002 is connected to the vaporizer electronics 402. The vaporizer electronics 402 control the dispenser 132. There is a reservoir 300 which feeds hyperpolarized contrast agent to the dispenser 132. Then finally the dispenser 132 is connected to a face piece 138.

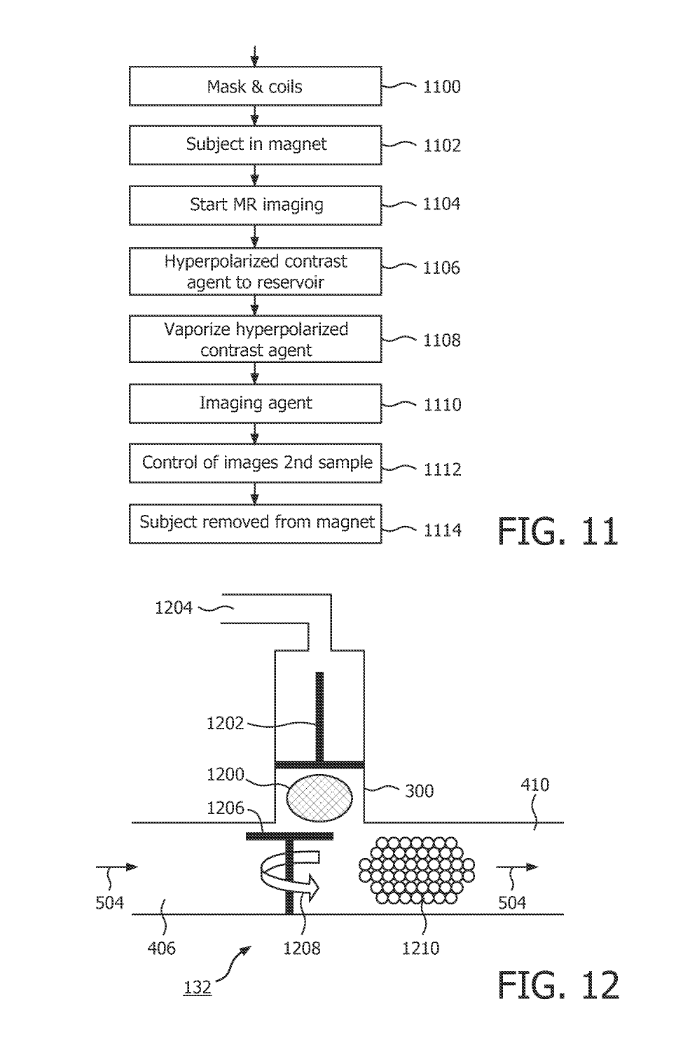

FIG. 11 shows an embodiment of a method for controlling the magnetic resonance imaging system for acquiring magnetic resonance imaging images when hyperpolarized vapor is administered to a subject. In step 1100 the face piece and coils are mounted on a subject. The dispenser is connected to the face piece. In step 1102 the subject is placed into the magnet. In step 1104 magnetic resonance imaging is started. At this step a proton image is prepared of the field of view. In step 1106 hyperpolarized contrast agent is placed in the reservoir or the dispenser. In step 1108 the hyperpolarized contrast agent is vaporized. In step 1110 magnetic resonance imaging of the hyperpolarized contrast agent is performed. In step 1112 the images are analyzed and a second sample of hyperpolarized contrast agent is administered to the subject. In step 1114 the subject is removed from the magnet.

FIG. 12 shows a dispenser 132 which uses a vaporizer that comprises a chopper adapted for chopping solid hyperpolarized contrast agent 1200 into particles 1210. There is a gas flow tube 406. There are arrows showing the direction of gas flow through the gas flow tube 406. Located within the gas flow tube 406 is a rotating chopper 1206. The arrow 1208 shows the direction of rotation of the rotating chopper 1206. The direction of the chopper may also be reversed. The rotating chopper 1206 chops solid hyperpolarized contrast agent 1200 into particles 1210 of hyperpolarized contrast agent. The particles of hyperpolarized contrast agent 1210 are small enough that they melt and are vaporized before they reach the respiratory system of the subject. The reservoir 300 for the solid hyperpolarized contrast agent 1200 is located directly above the rotating chopper 1206. There is an actuator 1202 for moving the hyperpolarized contrast agent 1200 into the rotating chopper 1206. There is a cooling system 1204 mounted adjacent to the actuator 1202 which is used to keep the solid hyperpolarized contrast agent 1200 in solid form. If toxic chemicals are incorporated into the solid hyperpolarized contrast agent, as was previously discussed, it would be beneficial to filter the hyperpolarized contrast agent before it reaches the respiratory system of the subject. Since the solid hyperpolarized contrast agent is not able to be filtered, the vapor produced by the melting of the particles 1210 should be filtered. This could be accomplished by placing a filter in either the gas flow tube of the dispenser, the attachment component, or be incorporated into the face piece.

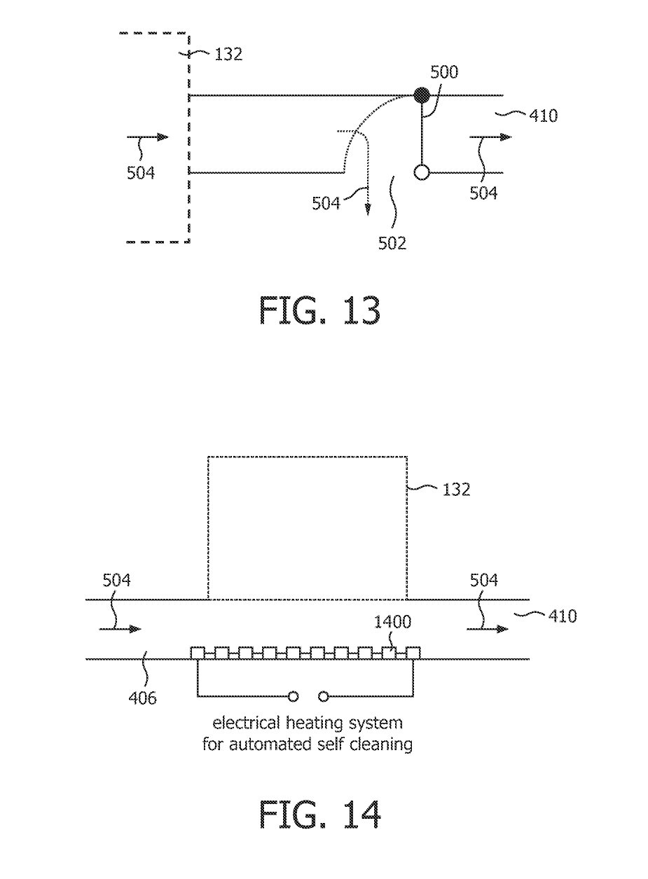

FIG. 13 shows the operation of the bypass valve 500. The bypass valve 500 is used during a self-cleaning procedure. Gas exits the dispenser 132. The possible directions of gas flow are shown with the arrows labeled 504. In this figure the valve is in a position such that there is no gas flow to the face piece and all of the gas is directed to a cleaning outlet 502 using high-pressure cleaning

FIG. 14 shows an electrical heating system 1400 which is used to reduce condensation in the flow tube 406. Arrows 504 indicate the direction of travel of gas through the gas flow tube 406. The dispenser 132 used to vaporize the hyperpolarized contrast agent is shown above the electrical heating system 1400. When the electrical heating system is turned on this will cause any hyperpolarized contrast agent which has condensed on the gas flow tube 406 to re-vaporize. The electrical heating system can also be turned on to prevent condensation on the gas flow tube. This is particularly advantageous for the embodiment shown in FIG. 13 where frozen particles of the hyperpolarized contrast agent are shown. As the cold temperature of the particles of hyperpolarized contrast agent are likely to cause localized cooling of the gas flowing through the gas flow tube.

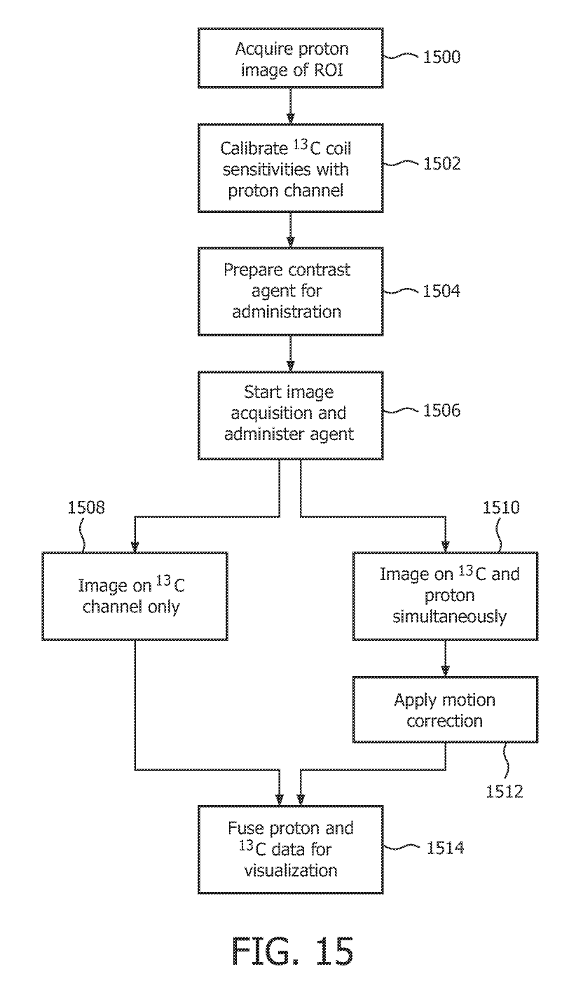

FIG. 15 shows a method of acquiring a magnetic resonance imaging image according to an embodiment of the invention. In step 1500 a proton image of a region of interest is acquired. In step 1502 a coil adapted for receiving the MRI signal of the hyperpolarized contrast agent is calibrated using the proton image that was acquired in step 1500. In step 1504 the hyperpolarized contrast agent is prepared for administration. In step 1506 the MRI image is acquired and the hyperpolarized contrast agent is administered. The method then branches into two possible variations. In a first variation in step 1508 only the magnetic resonance imaging data for the hyperpolarized contrast agent is acquired. Then in step 1514 the data for the proton image that was acquired in step 1500 and the magnetic resonance imaging data acquired in step 1508 is fused to visualize both the hyperpolarized contrast agent and the proton image data. In another alternative to this is that the hyperpolarized contrast agent and the proton data are imaged simultaneously in step 1510 immediately after step 1506. Then in step 1512 such things as motion correction are then applied, this could be such things as using a navigator to monitor breathing motion or for triggering during a certain phase of the heartbeat. Since the signal from the hyperpolarized contrast agent would likely be much less than the proton signal imaging the proton signal simultaneously would be advantageous for performing the motion correction. Then in the step after 1512 the proton MRI imaging data and the MRI data for the hyperpolarized contrast agent are then combined for visualization.

### LIST OF REFERENCE NUMERALS

100 Magnetic resonance imaging system
102 magnet
104 First coil
106 Second coil
108 Radio frequency transceiver
110 Magnetic field gradient coil
112 Magnetic field gradient coil power supply
114 Subject
116 Subject support
118 Region of interest
120 Computer system
122 Hardware interface
124 User interface
126 Microprocessor
128 Computer program product
130 Model module
132 Dispenser
134 Cartridge
136 Attachment component
138 Face piece
140 Region with a location fixed to the magnetic field of 102
300 Reservoir
302 Gas inlet
304 Hyperpolarized contrast agent
400 Electrical control
402 Electronics for controller and sensors
404 Valve
406 Gas flow tube
408 Liquid flow tube
410 Outlet to attachment component
412 Nozzle
414 support
500 Bypass valve
502 Cleaning outlet
504 Arrow showing direction of gas flow
506 Arrow showing amplitude of vibration
508 Droplets
510 Piezoelectric actuator
600 Cover
602 Support for piezoelectric actuator
606 Vibrating nozzle
700 Cartridge holder
702 Direction of rotation
900 Cartridge holder
1000 Console
1002 Magnetic resonance imaging spectrometer
1004 Magnetic resonance imaging magnet and magnetic field gradients
1200 Frozen hyperpolarized contrast agent
1202 Actuator
1204 Cooling system
1206 Rotating chopper
1208 Direction of rotation
1210 Frozen particles
1400 Electric heating system

1. A dispenser for dispensing a hyperpolarized vapor to a subject during a magnetic resonance imaging examination, the dispenser comprising:
an attachment component for a face piece adapted to receive a surface of the subject such that when the subject inhales the hyperpolarized vapor enters the respiratory system of the subject,
a reservoir adapted for receiving a hyperpolarized contrast agent,
a gas flow tube connected to the attachment component,
a vaporizer for vaporizing the hyperpolarized contrast agent in the gas flow tube into the hyperpolarized vapor, and
a controller for controlling when the vaporizer vaporizes the hyperpolarized contrast agent.

2. The dispenser of claim 1, wherein the dispenser further comprises a nuclear magnetic resonance coil adjacent to the reservoir, wherein the nuclear magnetic resonance coil is adapted for measuring the degree of polarization of the hyperpolarized contrast agent when connected to a nuclear magnetic resonance apparatus.

3. The dispenser of claim 1, wherein the reservoir is adapted for storing the hyperpolarized contrast agent in liquid form.

4. The dispenser of claim 1, wherein the reservoir is adapted for storing the hyperpolarized contrast agent in solid form, wherein the dispenser further comprises a heater adapted for liquefying the hyperpolarized contrast agent before vaporization.

5. The dispenser of claim 4, wherein the heater is adapted for melting the hyperpolarized agent at a rate equal to the rate at which it is vaporized.

6. The dispenser of claim 1, wherein the dispenser further comprises a breathing sensor adapted for detecting inhalation and exhalation by the subject, wherein the controller is adapted for controlling the vaporizer on the basis of the detected inhalation and exhalation such that the hyperpolarized contrast agent is vaporized only during inhalation by the subject.

7. The dispenser of claim 1, wherein the controller is adapted for receiving instructions from a computer system of a magnetic resonance imaging system, wherein the computer system is adapted for instructing the controller when to allow the vaporizer to vaporize the hyperpolarized contrast agent.

8. The dispenser of claim 1, wherein the reservoir is adapted for storing the hyperpolarized contrast agent in solid form, wherein the vaporizer comprises a chopper adapted for chopping the hyperpolarized contrast agent into particles small enough that the particles melt and are vaporized before being inhaled by the subject.

9. The dispenser of claim 1, wherein the reservoir is adapted for receiving a cartridge containing the hyperpolarized contrast agent.

10. The dispenser of claim 1, wherein the dispenser further comprises a cartridge holder adapted for holding at least two cartridges, and wherein the cartridge holder is adapted for automatically changing which cartridge is mounted in the reservoir.

11. The dispenser of claim 1, wherein the dispenser further comprises a bypass valve adapted for selecting if gas flowing through the gas flow tube is directed to the face piece or is directed to a cleaning outlet, wherein the control system is adapted for controlling the bypass valve in order to directing the gas flow to the cleaning outlet during a self cleaning procedure.

12. The dispenser of claim 1, wherein at least one portion of the gas flow tube has an electric heater adapted for re-vaporizing condensate of the hyperpolarized vapor.

13. A magnetic resonance imaging system adapted for acquiring magnetic resonance image images comprising:
a magnet for generating a magnetic field for orientating the magnetic spins of nuclei,
a radio frequency system comprising a first coil calibrated for acquiring magnetic resonance imaging data of protons and a second coil calibrated for acquiring magnetic resonance imaging data for the hyperpolarized contrast agent,
a magnetic field gradient coil for spatial encoding and manipulating the orientation of the magnetic spins of nuclei,
a magnetic field gradient coil power supply for supplying current to the magnetic field gradient coil,
a computer system for constructing images from the magnetic resonance imaging data and for controlling the operation of the magnetic resonance imaging system.
a dispenser according to claim 1.

14. The magnetic resonance imaging system of claim 13, wherein the reservoir of the dispenser is mounted with a location fixed relative to the magnetic field of the magnet, and wherein the location is chosen such that the magnetic field of the magnet preserves the hyperpolarization of the hyperpolarized contrast agent.

15. A method of acquiring a magnetic resonance imaging image of a subject using a magnetic resonance imaging system according to claim 15, the method comprising:
acquiring a first set of magnetic resonance imaging data of a region of interest of the subject using the first coil,
calibrating the second coil with the computer system using the first set of magnetic resonance imaging data and a model,
administering hyperpolarized vapor to the subject using the dispenser,
acquiring a second set of magnetic resonance imaging data image in the region of interest using the second coil, and
constructing a magnetic resonance imaging image from the second set of magnetic resonance imaging data using the computer system.
